(19) United States
(12) Patent Application Publication
(10) Pub. No.: US 2021/0007602 A1
(43) Pub. Date: Jan. 14, 2021

(54) BRAIN IMPLANT WITH SUBCUTANEOUS WIRELESS RELAY AND EXTERNAL WEARABLE COMMUNICATION AND POWER DEVICE

(71) Applicant: Neuralink Corp., Fremont, CA (US)

(72) Inventors: Dongjin Seo, San Francisco, CA (US); Max J. Hodak, San Francisco, CA (US); Vanessa M. Tolosa, Emeryville, CA (US)

(73) Assignee: Neuralink Corp., Fremont, CA (US)

(21) Appl. No.: 16/926,420

(22) Filed: Jul. 10, 2020

Related U.S. Application Data
(60) Provisional application No. 62/873,745, filed on Jul. 12, 2019.

(51) Int. Cl. A61B 5/00; A61B 5/04

(52) U.S. Cl. CPC A61B 5/0031; A61B 5/04001; A61B 5/6864; A61B 2562/0209; A61B 5/0028; A61B 5/0017; A61B 5/0006

(57) ABSTRACT

A brain-machine interface (BMI) is described in which many flexible electrodes for implanting within a subject's brain run to a cylindrical sensor device configured to fit inside a burr hole in the cranium. The devices contain sealed electronics that convert analog neural voltages to digital signals, or vice versa, and connects through a serial cable to a subcutaneous relay on the mastoid region (behind the subject's ear) or other suitable location. The relay draws power from and communicates with an externally worn device and distributes the power to the devices. The externally worn device communicates wirelessly or through a tether to a base station computer for data analysis and/or stimulation.

### CROSS-REFERENCES TO RELATED APPLICATIONS

This application claims the benefit of priority of U.S. Provisional Application No. 62/873,745 titled “Brain Implant with Subcutaneous Wireless Relay and External Wearable Communication and Power Device” and filed on Jul. 12, 2019, which is herein incorporated by reference in its entirety for all purposes.

### STATEMENT AS TO RIGHTS TO INVENTIONS MADE UNDER FEDERALLY SPONSORED RESEARCH AND DEVELOPMENT

Not Applicable

### BACKGROUND

Brain-machine interfaces (BMIs) hold tremendous promise for the restoration of sensory and motor function and the treatment of neurological disorders. Most of these applications require fine scale communication—at the level of individual neurons—with large numbers of neurons across multiple brain areas. There are not yet any clinically translatable approaches for achieving this level of brain interfacing.

Microelectrodes are the most common and effective tool for recording the activity of single neurons in the brain, but there has been little practical technology for stably recording activity across very large numbers of neurons in targeted brain areas.

Currently, almost all devices for long-term neural recording are made from rigid metals or semiconductors, in part because these materials are stiff enough to penetrate into the brain. This stiffness can cause problematic chronic motion between the electrodes and the brain. The motion, combined with the large size of most electrodes relative to the scale of brain cells, leads to local tissue damage and/or immune response that eventually degrades recordings. Moreover, the manual insertion of electrodes is painstaking, making it impractical to implant large numbers of probes in a brain.

Advanced neural interfaces will require increasing the number of accessible neurons by many orders of magnitude than what is available by current methods. Further, it can be important that these neurons be in diverse parts of the brain. For example, densely sampling 100,000 neurons in a single cortical column is much less information-rich than sampling pockets of 10,000 neurons across ten different brain areas of interest. And reading out information from all of these neurons can be problematic. There is a large amount of information in the form of currents and/or voltages that needs processing and communication to the outside world.

There is a need in the art for better BMIs.

### BRIEF SUMMARY

Generally, a brain electrode implant system is described that includes one or more cylindrical sensors that fit flushly into burr holes in a subject's skull with wires that lead to a subcutaneous relay/router device behind the subject's ear. The relay communicates inductively with a device worn by the subject, which in turn communicates wirelessly or otherwise with a base station.

Each sensor nestled within its burr hole receives signals from a microfabricated polymer cable embedded with hundreds or thousands of metal electrodes descending into the subject's brain. The electrodes are implanted using a specialized surgical robot that pulls each electrode with a rigid needle into the brain during surgery before pulling out and pulling in another electrode. The received electrical signals, analog voltages and currents, are converted onboard the sensor to digital and then assessed. If an electrical signal denotes a neural spike or other interesting event, then it is multiplexed with other events and fed through a serial cable to the relay. The relay then transmits it offboard to the base station.

Generally, embodiments of the present disclosure relate to a brain implant communication relay apparatus comprising electrodes connected with conductive traces embedded in a flexible polymer ribbon, a biocompatible housing into which the conductive traces extend, an analog-to-digital converter (ADC) within the housing and connected with the conductive traces through a hermetically sealed feedthrough, a multiplexer within the housing and connected with the ADC, the multiplexer configured to multiplex together digital signals from the ADC, a serial communications wire operatively connected with the multiplexer, a subcutaneous wireless relay configured for surgical attachment to a mastoid process or other part of a subject, the relay connected with the serial communications wire, and an external wearable communications device configured to wear behind an ear and inductively or optically communicate through skin with the relay.

The multiplexer can include a demultiplexer configured to demultiplex serial signals from the serial communications wire into individual digital signals for the ADC for output to the electrodes.

The apparatus can include a primary power coil within the external wearable communications device, and a secondary power coil within the subcutaneous wireless relay. Alternatively or in addition, the apparatus can include an optical or ultrasonic power transmitting device within the external wearable communications device, and an optical receiving device within the subcutaneous wireless relay.

The apparatus can include a base station configured to operatively connect with the external wearable communications device. It can include an amplifier within the housing and connected with the ADC. The biocompatible housing can have a height less than 10 millimeters (mm). The biocompatible housing can have a cylindrical portion configured to fit within a burr hole in a cranium.

### BRIEF DESCRIPTION OF THE DRAWINGS

FIG. 1A is a perspective top-rear view of brain-machine interface (BMI) implants and a relay in accordance with an embodiment.

FIG. 1B is a close up view of a brain implant of FIG. 1A.

FIG. 2A is an illustrative side view of a human head with BMI implants connected with a common cable in accordance with an embodiment.

FIG. 2B is a perspective top view of an implant of FIG. 2A.

FIG. 2C is a perspective top view of the implant of FIG. 2B with its cover removed.

FIG. 3 is an exploded view of an implant of FIG. 1A.

FIG. 4 is a cross section view of implanted electrodes running to a BMI implant in accordance with an embodiment.

FIG. 5 illustrates a base station in accordance with an embodiment.

### DETAILED DESCRIPTION

A brain electrode implant system is described that may be composed of multiple brain-machine interface (BMI) implants. Each implant, which can have a diameter of up to 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 20 mm, 25 mm, or 30 mm, is referred to as “sensor.” It fits into respective diameter burr holes in a subject's skull. A burr hole covers secure the sensors.

Each sensor device contains custom, low-power integrated circuit (IC) chips for onboard amplification and digitization. The sensor gathers data from flexible electrodes that have been implanted below the burr hole into the brain. Tunneling wires are routed from the sensor under the scalp to a subcutaneous relay/router element behind a subject's ear, which communicates and receives power wirelessly (e.g., inductively) with an externally worn pod device. The sensors and electrodes are implanted via assistance from a neurosurgical robot.

Electrical signals within a subject's brain are picked up by thin-film electrodes and transported to an electronic sensor set in burr holes of the subject's skull. The pill-shaped sensor includes amplifiers, analog-to-digital converters (ADC), and multiplexing electronics to turn the brain signals into timestamped, serialized digital packets. The serialized packets from the sensor and other sensors are set through subcutaneous wires underneath the scalp to a relay device under the skin in the mastoid region (behind the subject's ear) or another suitable location. The relay device sends signals to, and is powered through the skin by, an external device worn behind the subject's ear. The relay device and external worn device send and receive power and communications through magnetic induction coils or optically. The external worn device then sends signals through wires, or wirelessly, to a base station.

Stimulation to areas of the brain may be driven through a communication path opposite to that of communicating signals out of the brain.

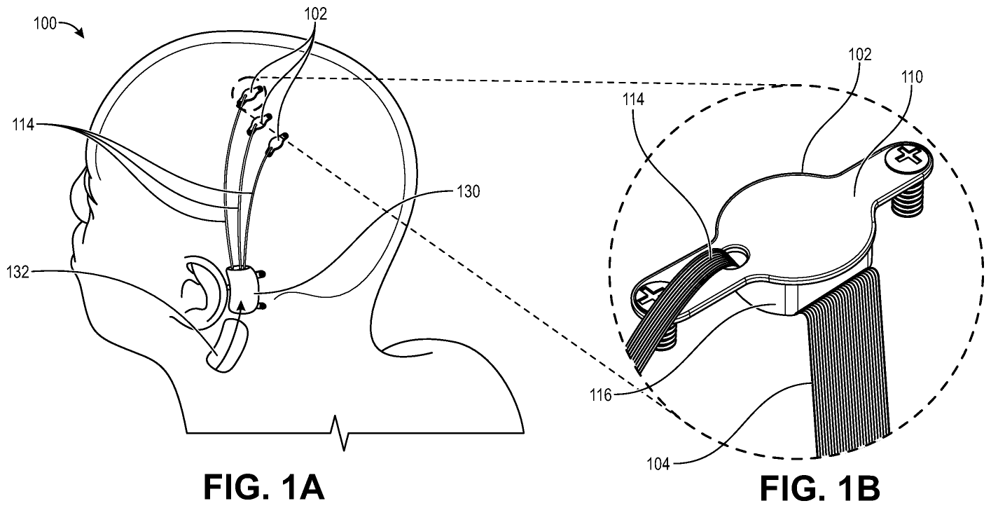

FIGS. 1A-1B are perspective views of brain-machine interface (BMI) implants and a relay on a subject. BMI system 100 includes cylindrical BMI implants 102 nestled within burr holes in the subject's cranium. Individual thin film serial cables run subcutaneously to relay 130, which has been surgically attached to the mastoid process of the subject. External pod 132 is worn by the subject directly over relay 130. Relay 130 communicates data with the external pod, essentially communicating the signals within the subject's brain to the outside world.

External, wearable pod 132 contains electronics and communicatively connects to a base. It is affixed with a biocompatible, water-resistant adhesive, positioned for optimal coupling with an implanted relay/router using a pair of magnets. The adhesive is disposable and can stay on the skin without causing irritation for multiple days. There are no magnets implanted under the skin so that, without the pod, the implanted system is magnetic resonance imaging (MM) compatible.

Power is delivered using near-field magnetic induction (or optically or ultrasonically), from the externally worn device, and the base station, that contains a power source to the implanted relay/router. The power source can be a battery, outlet, or other source. The inductive coil can be housed in the perimeter of the device's case.

The exemplary system can maintain high total system power efficiency over 2-10 mm of transmission distance, especially with the addition of a third coil embedded in the base to boost the range. Its power amplifier can be capable of driving the coil at 8 MHz or other frequencies and delivering up to 200 mW or more. In order to ensure the safe RF power limits outlined by the U.S. Food and Drug Administration (FDA) are met, closed-loop, automatic power adjustments are possible via the radio link polling for sensor current draw from the implanted relay/router and controlling the drive strengths in the power amplifier. This can be managed by the microprocessor in the radio chip or additional programmable logic devices.

A commercial-off-the-shelf 900 MHz, or other frequency, TX/RX (transmit/receive) radio can be used for both uplink (relay/router-to-external device) and downlink (device-to-relay/router) communication. It can also be done optically with photodiodes and photodetectors. In order to ensure safety:
The radio in the external device serves as the master (e.g., radio in the relay/router is the slave)
The radio operates in a half-duplex mode (e.g., TX & RX will not turn on at the same time)
Transmission reliability is implemented by end-to-end applications (e.g., the sensor and an external device) while the radio link provides a best-effort (user datagram protocol (UDP) style) transparent communication
The radio manages the following tasks all in parallel using dual on-chip ARM processors:
   Data transmission
   Data reception
   Self-diagnosis (power monitoring, watchdog, . . . )
   Radio management (authentication, control, log, flash update, . . . )
   Radio sleep mode control for power saving

In the exemplary embodiment, the radio has a dual ARM core for managing radio tasks, control flow, self-diagnosis, and power monitoring, all in parallel. There is an additional field-programmable gate array (FPGA) on the router to manage communications with each sensor. Upon power-up, the system resets, then initialize to a known state. Firmware upgrades to the relay/router can require new cryptographically-signed firmware to be programmed from the pod.

Upon power-up of the system, downlink communication initializes and configures the implanted system (implanted relay/router and sensor). Then, in a typical operating condition, the relay/router radio initiates uplink communication in order to stream recorded neural data out to the link. Given sufficient link margin and omni-directional antenna patterns of the 900 MHz chip antenna, the radio link is robust to misalignment and changes in the operating condition. There is a simple handshaking protocol and CRC (cyclic redundancy check) packets in the payload between the two radios to detect packet loss in the link. In the event that pod needs to reconfigure the implanted system parameters or update firmware, radio prioritizes command from the pod over other processes to execute.

FIG. 1B shows more detail of one of the BMI implants 102. Serial cable 114 exits implant 102 out the top and over burr hole cover 110. Beneath burr hole cover 110 is biocompatible housing 116, which has cylindrical portions designed to fit within the burr hole. At the bottom of the housing, flexible polymer ribbon cable 104 exits BMI implant 102, and its polymer-embedded electrodes descend into the brain.

The burr holes are about 8 millimeters in diameter and drilled using specialized surgical tools. During surgery, thin film electrodes, sometimes numbering in the hundreds or thousands, are delicately inserted into the cortex at precise locations to avoid vasculature.

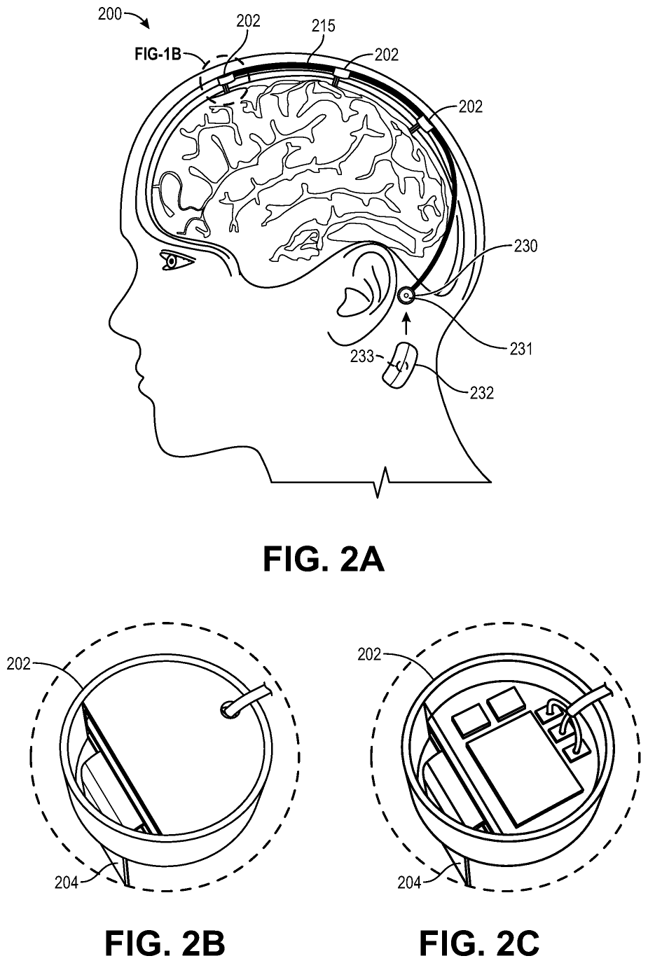

FIG. 2A illustrates a human head with alternative system 200 of three brain-machine interface (BMI) implants 202 set within holes in the subject's cranium (skull bone). The implants are located in different lobes, or areas of the brain, to capture or stimulate targeted sections. The thin film electrodes merge into ribbon cable 204 at one end, which in turn is preconnected to the implant. Each implant is carefully set on top of the ribbon cable to cover the burr hole.

Physically, the sensor is a silicone- or other polymer-casted package that is molded into a cylindrical puck with a thin film of electrodes emerging from one end, and the wires to connect to the implanted router from the other. Inside the puck is a hermetic glass or other biocompatible material package containing electronics and thin film attached through vias or otherwise to a custom system on a chip (SoC).

In the exemplary embodiment, each sensor pill has 1,024 channels. Each array has 64 threads of 16 electrodes each. Every channel supports both stimulation and recording. The sensor's physical package is an 8 mm cylindrical “puck” that fits into an 8 mm drilled burr hole to sit flush with the surface of the skull.

The sensor implants connect through daisy chain serial cable 215 to relay 230. It communicates with and is powered by external pod 232. Primary coil 233 in pod 232 powers secondary coil 231 in relay 230 through electromagnetic induction.

In the exemplary system, there are no radios and batteries in the implant. If a patient takes off the pod device, then the implant loses power and he or she disconnects his or her implant from the outside. In an alternative embodiment, a small battery can be included in the implant.

FIGS. 2B-2C show an implant with a ribbon cable extending below it. FIG. 2C shows the implant without its top cover. Within each implant is circuitry, including integrated circuit (IC) chips, capacitors, and other components. The ICs receive from, and/or transmit to, the thin film electrodes that are surgically implanted within the subject's cranium. The ICs can include analog-to-digital converters (ADC) and/or digital-to-analog converters (DAC) in order to convert analog signals in the brain to or from digital signals of a computer.

The IC chips that include the ADCs can also include multiplexers/demultiplexers that multiplex digital signals together to put on the serial cable, or demultiplex serial signals from the serial cable apart for output to the DACs. The former is for reading out from the brain, while the latter is for stimulating the brain.

Sitting in the burr hole, within the biocompatible housing, are a tight pack of electrical components. The components are carefully positioned to interface with the thin film ribbon cable of what may be thousands of individual, electrically isolated electrodes.

The leads and electrodes are manufactured using microelectromechanical systems (MEMS) technologies. The leads are manufactured monolithically with the hermetic package substrate and connect with electrodes. Each electrode site is oval and has a geometric surface area of ˜370 μm² that is coated with a high surface area material.

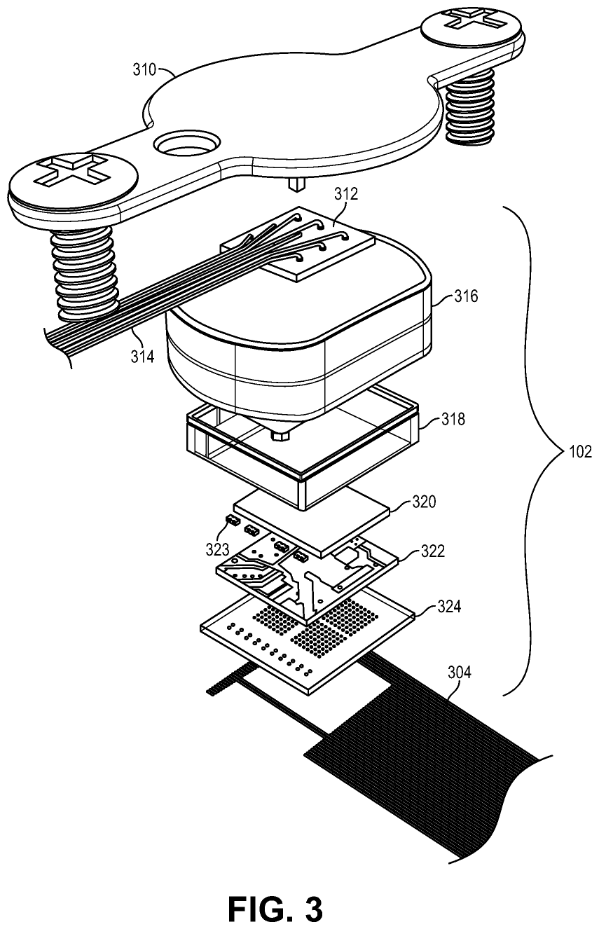

FIG. 3 is an exploded view of implant 102 and shows additional elements, such as burr hole cover 310 and additional packaging. The total height of the assembly fits within the average skull thickness of humans, which is about 6.5 mm for males and about 7.1 mm for females. After the assembly is surgically implanted in a burr hole, burr hole cover 310 is screwed to the cranium.

At the top of implant 102, just under burr hole cover 310, is serial cable 314. The cable may run to a different implant or to a relay with several digital lines. In either case, cable 314 sends output from analog-to-digital converters (ADC)s in the IC chip below to other systems or bring input from those same systems to the chip for commands, processing, or stimulation. Cable 314 connects to hermetic seal 312.

Hermetic seal 312 electrically connects the cable through outer glass housing 316. Glass housing 316 protects hermetically sealed walled housing 318. It is this inner walled housing that encases the IC chip.

Hermetically sealed walled housing 318 covers IC chip 320, capacitors 323, and PC board 322. In the exemplary embodiment, housing 318 is approximately 5 millimeters (mm) in width and breadth and about 2-3 mm in height. Housing 318 is laser sealed against hermetic feedthrough 324, encasing the IC chip and other components. The empty volume inside hermetically sealed walled housing 318 that is not occupied by components may be encapsulated in an epoxy & silicone overmold.

Hermetic feedthrough 324 electrically connects components within housing 318 to a thin film flexible cable, otherwise called ribbon cable 304. This flex cable can have hundreds to thousands of conductive traces within it leading to individual electrodes. Built up directly on the glass of the hermetic feedthrough, it can be referred to as “monolithically formed” with the hermetic feedthrough. In the figure, ribbon cable 304 projects laterally from the bottom of the assembly. However, it turns downward when implanted, as shown in FIG. 2C.

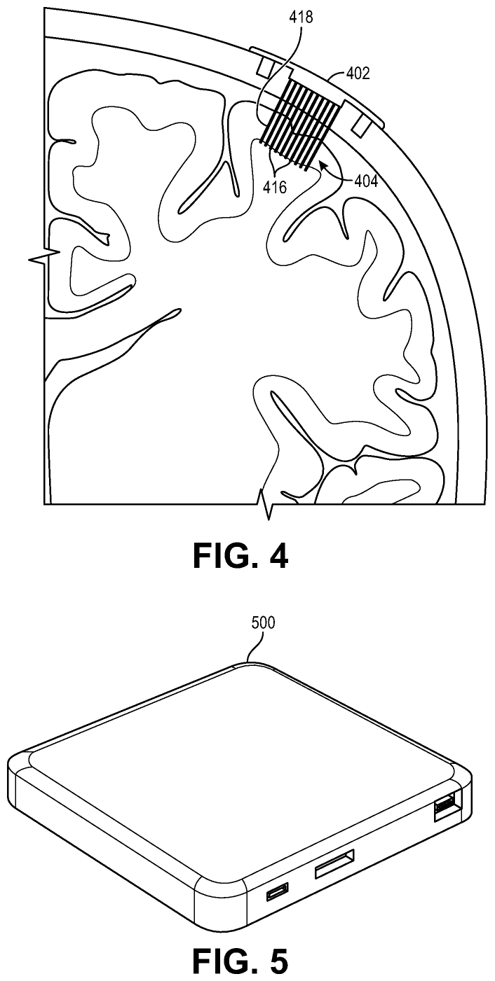

FIG. 4 is a cross section view of implanted electrodes in accordance with an embodiment. BMI implant 402 connects with thin film cable 404.

Thin film cable 404 comprises electrodes 416 embedded in flexible polymer ribbon 418. Thin film cable was manufactured using additive and subtractive microfabrication techniques, such as electroplating and photolithography. Thus, its electrodes may have a rectangular cross section. The electrodes can be extremely small, on the order of tens or hundreds of microns in effective diameter.

While joined at the upper end, thin film cable splits into separate insulated wires, or threads, before descending into the brain. These different threads may have multiple electrodes along their lengths that probe at different depths of the brain.

FIG. 5 illustrates base station 500. Base station 500 communicates with an externally worn pod, such as pod 232 (see FIG. 2), either through a wire or wirelessly. The pod may be swapped out to recharge, while the base station may be plugged in to a power outlet. The base station may log and process data itself, or it can connect with a personal computer, smart phone, or other computing device.

In some embodiments, the pod contains a Bluetooth® radio, which pairs to an iOS or Android app on a mobile phone that allows the patient to control non-safety-critical settings, receive and manage over-the-air firmware updates, and follow an in-app training program for learning to control and use the implanted neural interface.

Once configured, the pod may show up as a Bluetooth® connected keyboard and mouse that are pairable by any computer capable of using a regular Bluetooth® keyboard and mouse. The neural signals to HID control model is run on the pod, so there is no special software that must be installed on the receiving computer, which only receives ordinary keyboard/mouse control input.

It should be appreciated that a brain implant or other system and a respective control system for the brain implant can have one or more microprocessors/processing devices that can further be a component of the overall apparatuses. The control systems are generally proximate to their respective devices, in electronic communication (wired or wireless) and can also include a display interface and/or operational controls configured to be handled by a user to monitor the respective systems, to change configurations of the respective systems, and to operate, directly guide, or set programmed instructions for the respective systems, and sub-portions thereof. Such processing devices can be communicatively coupled to a non-volatile memory device via a bus. The non-volatile memory device may include any type of memory device that retains stored information when powered off. Non-limiting examples of the memory device include electrically erasable programmable read-only memory (“ROM”), flash memory, or any other type of non-volatile memory. In some aspects, at least some of the memory device can include a non-transitory medium or memory device from which the processing device can read instructions. A non-transitory computer-readable medium can include electronic, optical, magnetic, or other storage devices capable of providing the processing device with computer-readable instructions or other program code. Non-limiting examples of a non-transitory computer-readable medium include (but are not limited to) magnetic disk(s), memory chip(s), ROM, random-access memory (“RAM”), an ASIC, a configured processor, optical storage, and/or any other medium from which a computer processor can read instructions. The instructions may include processor-specific instructions generated by a compiler and/or an interpreter from code written in any suitable computer-programming language, including, for example, C, C++, C #, Java, Python, Perl, JavaScript, etc.

While the above description describes various embodiments of the invention and the best mode contemplated, regardless how detailed the above text, the invention can be practiced in many ways. Details of the system may vary considerably in its specific implementation, while still being encompassed by the present disclosure. As noted above, particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific examples disclosed in the specification, unless the above Detailed Description section explicitly defines such terms. Accordingly, the actual scope of the invention encompasses not only the disclosed examples, but also all equivalent ways of practicing or implementing the invention under the claims.

In some embodiments, the systems and methods of the present disclosure can be used in connection with neurosurgical techniques. However, one skilled in the art would recognize that neurosurgical techniques are a non-limiting application, and the systems and methods of the present disclosure can be used in connection with any biological tissue. Biological tissue can include, but is not limited to, the brain, muscle, liver, pancreas, spleen, kidney, bladder, intestine, heart, stomach, skin, colon, and the like.

The systems and methods of the present disclosure can be used on any suitable multicellular organism including, but not limited to, invertebrates, vertebrates, fish, bird, mammals, rodents (e.g., mice, rats), ungulates, cows, sheep, pigs, horses, non-human primates, and humans. Moreover, biological tissue can be ex vivo (e.g., tissue explant), or in vivo (e.g., the method is a surgical procedure performed on a patient).

The teachings of the invention provided herein can be applied to other systems, not necessarily the system described above. The elements and acts of the various examples described above can be combined to provide further implementations of the invention. Some alternative implementations of the invention may include not only additional elements to those implementations noted above, but also may include fewer elements. Further any specific numbers noted herein are only examples; alternative implementations may employ differing values or ranges, and can accommodate various increments and gradients of values within and at the boundaries of such ranges.

References throughout the foregoing description to features, advantages, or similar language do not imply that all of the features and advantages that may be realized with the present technology should be or are in any single embodiment of the invention. Rather, language referring to the features and advantages is understood to mean that a specific feature, advantage, or characteristic described in connection with an embodiment is included in at least one embodiment of the present technology. Thus, discussion of the features and advantages, and similar language, throughout this specification may, but do not necessarily, refer to the same embodiment. Furthermore, the described features, advantages, and characteristics of the present technology may be combined in any suitable manner in one or more embodiments. One skilled in the relevant art will recognize that the present technology can be practiced without one or more of the specific features or advantages of a particular embodiment. In other instances, additional features and advantages may be recognized in certain embodiments that may not be present in all embodiments of the present technology.

What is claimed is:

1. A brain implant communication relay apparatus comprising:
electrodes connected with conductive traces embedded in a flexible polymer ribbon;
a biocompatible housing into which the conductive traces extend;
an analog-to-digital converter (ADC) within the housing and connected with the conductive traces through a hermetically sealed feedthrough;
a multiplexer within the housing and connected with the ADC, the multiplexer configured to multiplex together digital signals from the ADC;
a serial communications wire operatively connected with the multiplexer;
a subcutaneous wireless relay configured for surgical attachment to a subject, the relay connected with the serial communications wire; and
an external wearable communications device configured to wear behind an ear and inductively, optically, or ultrasonically communicate through skin with the relay.

2. The apparatus of claim 1 wherein the multiplexer includes a demultiplexer configured to demultiplex serial signals from the serial communications wire into individual digital signals for the ADC for output to the electrodes.

3. The apparatus of claim 1 further comprising:
a primary power coil within the external wearable communications device; and
a secondary power coil within the subcutaneous wireless relay.

4. The apparatus of claim 1 further comprising:
an optical power transmitting device within the external wearable communications device; and
an optical receiving device within the subcutaneous wireless relay.

5. The apparatus of claim 1 further comprising:
a base station configured to operatively connect with the external wearable communications device.

6. The apparatus of claim 1 further comprising:
an amplifier within the housing and connected with the ADC.

7. The apparatus of claim 1 wherein the biocompatible housing has a height less than 10 millimeters (mm).

8. The apparatus of claim 7 wherein the biocompatible housing has a cylindrical portion configured to fit within a burr hole in a cranium.
